(19) United States
(12) Patent Application Publication
(10) Pub. No.: US 2019/0307919 A1
(43) Pub. Date: Oct. 10, 2019

(54) COATING FOR ARTIFICIAL MUSCLES AND ACTUATORS

(71) Applicant: Lintec of America, Inc., Richardson, TX (US)

(72) Inventors: Marcio Dias Lima, Richardson, TX (US); Sergey Li, Richardson, TX (US)

(73) Assignee: Lintec of America, Inc., Richardson, TX (US)

(21) Appl. No.: 16/339,880

(22) PCT Filed: Oct. 6, 2017
(86) PCT No.: PCT/US2017/055565

§ 371 Date: Apr. 5, 2019

Related U.S. Application Data
(60) Provisional application No. 62/405,138, filed on Oct. 6, 2016.

(51) Int. Cl. A61L 27/34; A61L 27/04; A61L 27/44; A61L 27/06; A61L 27/16; A61L 27/18; A61L 27/50

(52) U.S. Cl. CPC A61L 27/34; A61L 27/047; A61L 27/443; A61L 27/06; A61L 27/306; A61L 27/18; A61L 27/446; A61L 27/50; A61L 27/16

(57) ABSTRACT

An actuator device that includes at least one fiber, and at least one first coating is disclosed. The first coating encloses the at least one fiber. The actuator device may include a plurality of fibers and/or a conducting material. The coatings may enclose the plurality of fibers, or each individual fiber. The coatings may provide moisture protection, UV protection, saline protection, and oxidation protection. The coating may be thermally and electrically conducting or insulating, depending on the specific function and environment of the actuator device.

### CROSS-REFERENCE TO RELATED APPLICATIONS

This application claims priority to U.S. Provisional Application No. 62/405,138 filed on Oct. 6, 2016, the contents of which are incorporated by reference in their entirety.

### BACKGROUND OF INVENTION

Artificial polymer muscles lacking a protective layer are exposed to the environment. For example, nylon, a particularly useful artificial muscle material, may be susceptible to degradation in the presence of water. Over time, nylon artificial muscle fibers may fail in moist environments. Also, nylon may be sensitive to electromagnetic radiation exposure.

### SUMMARY OF INVENTION

In one aspect, embodiments of the invention relate to an actuator device that includes at least one fiber, and at least one first coating. The first coating encloses the at least one fiber.

In another aspect, the actuator device may include a plurality of fibers and/or a conducting material. The coatings may enclose the plurality of fibers, or each individual fiber in the bundle.

In accordance with embodiments disclosed herein, the coatings may provide moisture protection, UV protection, saline protection, and oxidation protection. The coating may be thermally and electrically conducting or insulating, depending on the specific function and environment of the actuator device.

Other aspects and advantages of the invention will be apparent from the following description and the appended claims.

### BRIEF DESCRIPTION OF DRAWINGS

FIG. 1 is a schematic in accordance with one or more embodiments of the invention;

FIG. 2 is a schematic in accordance with one or more embodiments of the invention

### DETAILED DESCRIPTION

Embodiments of the invention will now be described in detail with reference to the accompanying Figures. Like elements in the various figures may be denoted by like reference numerals for consistency. Further, in the following detailed description of embodiments of the present invention, numerous specific details are set forth in order to provide a more thorough understanding of the claimed subject matter. However, it will be apparent to one of ordinary skill in the art that the embodiments disclosed herein may be practiced without these specific details. In other instances, well-known features have not been described in detail to avoid unnecessarily complicating the description.

In general, embodiments of the invention relate to a thin, coating in an actuating artificial muscle to protect the artificial muscle and, in some cases, enhance the properties of the artificial muscle. In the embodiments disclosed herein, the artificial muscle actuators include one or more fibers that are thermally driven. In one or more embodiments, the actuators include a conducting material so that the actuation may be stimulated electrically. In other words, an applied voltage or current may provide the necessary temperature changes for actuation. Embodiments of the coating layer may protect the artificial muscle fiber, and may improve characteristics of the produced artificial muscle or actuator.

Embodiments of the invention include a coating incorporated into actuators that utilize non-coiled or coiled yarns or polymer fibers that may be either neat or include a guest. The term “artificial muscle fiber” is generically used herein to describe a nanofiber yarn and twisted polymer fibers or a collection (bundles) of nanofiber yarns and twisted polymer fibers that perform actuation such as those described in PCT/US2017/030199, the contents of which are hereby incorporated by reference.

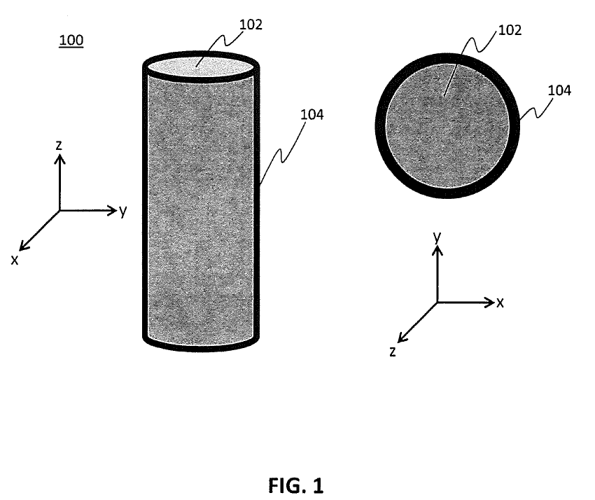

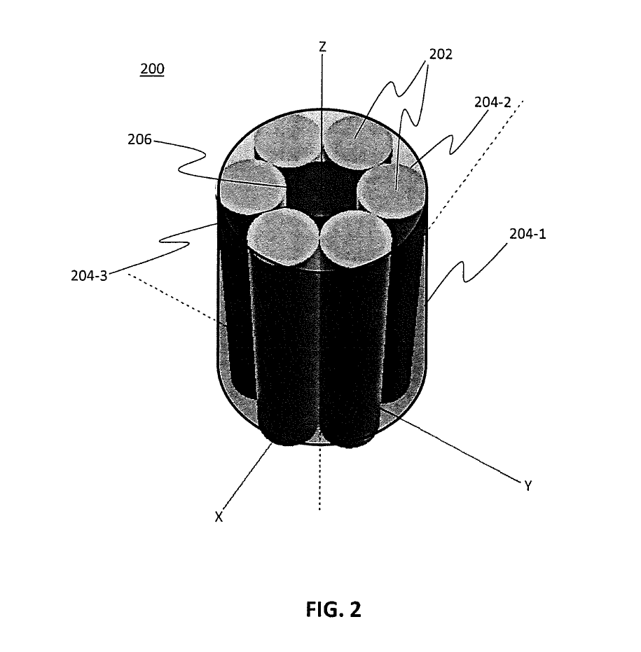

FIGS. 1 and 2 show schematics in accordance with one or more embodiments of the invention. FIG. 1 demonstrates a basic artificial muscle actuating fiber 100 that includes the fiber 102 with a coating 104 in accordance with embodiments disclosed herein.

For example, in one or more embodiments, a black colored coating can be applied so that the artificial muscle or actuator readily absorbs radiation. Such radiation may be used in the function of the actuator. In one more embodiments of the invention, a coating is selected that is suitable to interact closely with biological material.

As another example, in one or more embodiments the coating is reflective. A reflective muscle may be able to maintain exposure to the Sun without heating too far above the temperature of the surrounding environment.

In one or more embodiments, a coating may be thermally conducting. In such embodiments, the coating may enable heat to be more easily whisked away from the muscle fiber, which may improve stroke efficiency, and possibly prevent any defective spots from overloading with heat. Such “hot spots” may be caused by a conductor material in the artificial muscle or actuator having imperfections along the length of the artificial muscle fiber. If such hot spots are not addressed, there is a danger that the polymer fiber along that section will heat too high and melt resulting in a failure of the muscle.

In one or more embodiments, the coating may be thermally insulating. However, a thermal insulating coating can cause overheating the artificial muscle fibers. Therefore, in such embodiments, the coating may be thin (less than 5 microns) to prevent any overheating or degradation in the artificial muscle fiber actuator.

In one or more embodiments of the invention, the coating material is designed to lend new properties to the artificial muscle fiber. In one or more embodiments of the invention, the coating material is designed to protect the artificial muscle from environmental conditions. In some embodiments, the coating may serve to protect the conductor material and/or protect the polymer fiber.

In one or more embodiments of the invention, the coating may be multi-functional. For example, the coating may be designed to enhance the thermal properties, provide adhesion or reduce friction, and protect from, or incorporate into, the surrounding environment. Embodiments of the invention include multi-functional coatings that may be engineered for any combination of the above characteristics depending on the specific application for the artificial muscle actuator.

The coating may be designed to enhanced properties of the artificial muscle or actuator in accordance with embodiments disclosed herein. For example, the coating may be selected to interact well with biological material, making the artificial muscles useful for incorporation into devices in the human body. In these embodiments, care must be taken to ensure adequate thermal dissipation to prevent burn damage.

In one or more embodiments, the coating may provide electrical insulation to the conductor material and/or protect the polymer fiber. Such embodiments may be useful in artificial muscles that include a bundle of fibers forming the artificial muscle (or actuator).

For example, FIG. 2 is a schematic of a bundled fiber in accordance with one or more embodiments disclosed herein. The fiber bundle 200 includes a plurality of individual fibers 202. Each of the individual fibers may or may not include a coating 204-2. There may also be a coating 204-1 that encloses the plurality of individual fibers 202. As previously noted, the bundle may include a conductor material 206. The conductor material 206 may also have a coating 204-3. The coatings 204-1, 204-2, 204-3 may be different coatings selected based on the desired properties of the artificial muscle actuator.

In one or more embodiments, the coating may be designed to reduce surface friction. Such embodiments may also be useful in artificial muscles that include a bundle of fibers forming the artificial muscle (or actuator) as shown in FIG. 2. For example, the low surface tension of parylene as a coating material may increase slippage between the muscle fibers within a bundle. Such embodiments may be useful in creating tighter bundles of smaller fibers.

In one or more embodiments, the coating may be designed for protection from the environment. For example, moisture protection, UV radiation protection, oxidation protection, saline solution protection, and/or high temperature protection. Embodiments of the artificial muscle or actuator that include one or more metal wires may particularly benefit from saline protection. Embodiments that include high temperature protection may also protect the external environment from the high temperature of the conductive material, and/or protect the muscle fiber from sudden changes in external temperature.

Embodiments of the coating disclosed herein may be designed based on the thermal emissivity. For example, the coating may be designed to enhance the thermal emissivity. In such examples, the coating may be a black coating, or may be a paint-type coating with a known emissivity. The emissivity of nylon, which may be present in the artificial muscle fiber, is 0.85. In some embodiments, the coating may be designed to have an emissivity greater than the emissivity of the artificial muscle fiber. Increasing the thermal emissivity through the use of the coating may increase the efficiency of the artificial muscle actuator.

For example, a thermally conducting coating may prevent the formation of “hot spots” along sections of the artificial muscle length. Flaws in a conductor included in the artificial muscle and actuator may result in too much heat being applied at one area along the muscle. As a result, irreparable damage to the artificial muscle fiber may occur if the hot spot reaches too high a temperature. A thermally conducting coating may help dissipate the heat in these hot-spots.

In one or more embodiments of the invention, the structure of the coated artificial muscle fiber may be similar to that of a real muscle fiber in that there is a protective layer coating each muscle fiber that makes up the artificial muscle. In one or more embodiments, the protective coating may also be a layer coating the entire artificial muscle or actuator. In one or more embodiments, the coating may be uniform, with no punctures or defects that may allow the external environment to directly contact the artificial muscle fiber.

Artificial muscles or actuators may include a metal wire incorporated as a conductor material. In such embodiments, it may be advantageous for the protective coating to completely cover the metal wires. It may also be necessary that the metal wires do not separate from a surface of the fiber that makes up the artificial muscle or actuator. During the coating process, care must be taken in order to not insulate the metal wire from the surface of the fiber. Such insulation may negatively affect the performance of the artificial muscle fiber.

In one or more embodiments of the invention, a selective polyurethane coating may be used on metal wires included in the artificial muscle or actuator. For example, the conductive metal wire that is incorporated into the artificial muscle fiber may be pretreated with a polymer useful for coating the muscle fibers and the wire. Then, the polymer coating of the metal wire may be further melted to coat, or partially coat, the artificial muscle fiber. In such embodiments, the coating may be primarily deposited in areas close to the metal wires, leaving some areas of the polymer muscle fiber exposed. This selective coating may be useful in protecting the wires while intentionally leaving some of the muscle fibers exposed. In one or more embodiments, the selective coating may be used in combination with another coating layer, to provide greater protection for areas closer to the conductive wires.

Various polymers may be used for the coating, for example, parylene, polyurethane, polyvinyl based polymers, and fluorinated polymers in accordance with one or more embodiments disclosed herein. In one or more embodiments, the coating may be metal. For example, gold, silver, titanium, copper, nickel, and mixtures thereof may be used. In one or more embodiments, alloys of the above metals, or for example, chromium may be used. In one or more embodiments, a metal wire incorporated into the artificial muscle maybe coated with polyurethane. In one or more embodiments, the wire may be wrapped around the artificial muscle fibers and heated to melt the polyurethane to the muscle fiber surface. In such embodiments, more polyurethane may be added to completely coat the artificial muscle or actuator. In one or more embodiments, nano-composites, such as nanostructured clay in a polymer or graphene dispersed in a polymer, may be used as a coating material. Such embodiments may be advantageous for conducting heat and ensuring proper heat dissipation.

In general, the process for depositing the coating may include sputtering, electroplating, chemical vapor deposition (CVD), solution based deposition, and other techniques for producing a film or coating as known in the art. It may be necessary to coat the artificial muscle fibers after they have been twisted and/or coiled because the coating may be damaged in the twisting and/or coiling process. However, some embodiments may be coated prior to the twisting/coiling process. For example, silver coated nylon may be used in the artificial muscle fabrication to provide a coating incorporated prior to the twisting/coiling process.

In one or more embodiments, a polyurethane coated metal wire may be used as a conductor in the artificial muscle or actuator. The polyurethane on the wire may be further melted so that the polyurethane covers at least a portion of the artificial muscle fiber. Another coating of the same or different material may be subsequently applied onto the surface of the artificial muscle fiber in accordance with one or more embodiments.

It should be understood by those having ordinary skill that the present invention shall not be limited to specific examples depicted in the Figures and described in the specification. While the present invention has been described with respect to a limited number of embodiments, those skilled in the art, having benefit of this disclosure, will appreciate that other embodiments may be devised which do not depart from the scope of the invention as described herein. Accordingly, the scope of the invention should be limited only by the attached claims.

1. An actuator device comprising:
at least one fiber; and
at least one first coating, wherein the first coating encloses the at least one fiber.

2. The actuator device of claim 1, further comprising a plurality of fibers, wherein at least two of the plurality of fibers are coated by the first coating.

3. The actuator device of claim 2, wherein the plurality of fibers are enclosed by a second coating.

4. The actuator device of claim 3, wherein the second coating is biocompatible.

5. The actuator device of claim 3, further comprising:
a conducting material,
wherein the second coating protects the plurality of fibers and the conducting material from exposure to saline.

6. The actuator device of claim 4, wherein the first coating reduces surface friction between the plurality of fibers.

7. The actuator device of claim 1, further comprising a conducting material.

8. The actuator device of claim 7, wherein the conducting material is a metal wire, and the first coating coats the metal wire.

9. The actuator device of claim 7, wherein the conducting material is a metal wire, and a third coating coats the metal wire.

10. The actuator device of claim 9, wherein the third coating provides adhesion between the conducting material and the plurality of fibers.

11. The actuator device of claim 1, wherein the first coating is thermally insulating.

12. The actuator device of claim 1, wherein the first coating is thermally conducting.

13. The actuator device of claim 1, wherein the first coating is a black colored coating.

14. The actuator device of claim 1, where the first coating is reflective.

15. The actuator device of claim 1, wherein the first coating includes at least one of the following materials: parylene, polyurethane, gold, silver, titanium, copper, nickel, chromium, nanostructured clay in a polymer, graphene dispersed in a polymer, and fluorinated polymers.

16. The actuator device of claim 3, wherein the second coating includes at least one of the following materials: parylene, polyurethane, gold, silver, titanium, copper, nickel, chromium, nanostructured clay in a polymer, graphene dispersed in a polymer, and fluorinated polymers.

17. The actuator device of claim 9, wherein the third coating includes at least one of the following materials: parylene, polyurethane, gold, silver, titanium, copper, nickel, chromium, nanostructured clay in a polymer, graphene dispersed in a polymer, and fluorinated polymers.
